(19) United States
(12) Patent Application Publication
(10) Pub. No.: US 2024/0148661 A1
(43) Pub. Date: May 9, 2024

(54) HIGH TEMPERATURE RESISTANT PROBIOTICS FOR FOOD OR BEVERAGE AND METHOD OF MAKING THE SAME

(71) Applicant: Nano and Advanced Materials Institute Limited, Hong Kong (HK)

(72) Inventors: Joo Ann EWE, Hong Kong (HK); Chun Hay KO, Hong Kong (HK); Tsz Wai NG, Hong Kong (HK); Ka Man TSE, Hong Kong (HK); Mu WANG, Hong Kong (HK)

(21) Appl. No.: 18/499,252

(22) Filed: Nov. 1, 2023

Related U.S. Application Data
(60) Provisional application No. 63/421,575, filed on Nov. 2, 2022.

(51) Int. Cl. A61K 9/50; A61K 9/00; A61K 31/702; A61K 35/20; A61K 35/747

(52) U.S. Cl. CPC A61K 9/5073; A61K 9/0056; A61K 9/0095; A61K 9/5015; A61K 9/5026; A61K 9/5036; A61K 31/702; A61K 35/20; A61K 35/747; A61K 2035/115

(57) ABSTRACT

A heat and acid resistant probiotics microsphere having a size from 20 to 250 μm that can readily be incorporated into food or beverages that subsequently undergo thermal treatment. The synbiotic core includes a seed layer formed from at least one polysaccharide. A probiotic microorganism is coated on the seed layer. An acid-resistant shell layer is positioned over the synbiotic core, the acid-resistant shell layer comprising one or more pH-responsive polymers. A heat-resistant bilayer shell is positioned over the acid-resistant shell layer, the heat-resistant bilayer shell including an inner shell layer and an outer shell layer, wherein the inner shell layer includes a heat-resistant liposome layer and the outer layer includes a heat-resistant disaccharide or polysaccharide.

### CROSS-REFERENCE TO RELATED APPLICATION

The present application claims priority from a U.S. provisional patent application Ser. No. 63/421,575 filed Nov. 2, 2022, and the disclosures of which are incorporated by reference in their entirety.

### FIELD OF THE INVENTION

The present invention relates to a healthy food or beverage for delivering heat sensitive nutrients to a consumer in a food or beverage that is usually heat-processed. More specifically, the present invention relates to encapsulated probiotics with coating compositions to protect probiotics from high temperatures during industrial processing, while maintaining a sufficient number of live probiotics upon subsequent ingestion, surviving the gastrointestinal digestive environment.

### TECHNICAL BACKGROUND OF THE INVENTION

In this era, dysbiosis, an imbalance in the microbiota resulting in the distress in the functioning and metabolism of the microbiome, may have resulted in lower immunity and high susceptibility to infections of the human hosts (Xu et al. 2020), and may manifest in form of various illnesses. Dysbiosis is most commonly observed to occur in the gastrointestinal tract, which may, in part, cause diseases and conditions including but not limited to gastrointestinal symptoms (vomiting, diarrhea, abdominal, malnutrition, etc), Crohn's diseases, inflammatory bowel disease and Type 2 Diabetes.

However, such adverse conditions could be remedied through rebiosis, the process in which the native microbiota is re-established through therapeutic tools including probiotics, defined by the World Health Organization as “live microorganisms which when administered in adequate amounts confer a health benefit on the host”. As such, probiotics are gaining popularity across the globe, and it is predicted that the sales of probiotics-related products will continue to substantially increase worldwide. Studies show that a majority of the population would choose food and/or beverages as preferred probiotics delivery formats, compared to pills or tablets. Nevertheless, supplements in the form of pills or tablets are the dominant probiotics product in the current market. The consumption of these supplements has always been a struggle for the elderly and children because they may have difficulties in swallowing and “pill fatigue” psychology. Perceived as a more natural way of receiving a daily dose, incorporation of probiotics into food products may be a means to overcome the perception of probiotics as a prescription or medication. In addition, the increasing health awareness of public has also created a demand for multi-functional healthy food. Thus, there is a need for the development of novel probiotics food and beverages to fulfill the demand.

Conventionally, probiotics have been incorporated into milk products for fermentation to produce yoghurt, for example. The optimum growing condition of the starter cultures allows the probiotics to survive in the product; however, it must be refrigerated to prevent product spoilage resulting in a short product shelf life. The need for refrigeration has restricted the variety of probiotics products. In recent years, there has been a substantial increase in shelf-stable functional food and beverage industries. High temperature processing is common in the industry, which poses a limitation to incorporating probiotics in food and beverages. This is due to the fact that the viability of probiotics is susceptible to heat stress during manufacturing of processed foods. With the limitation of current technology, infusion of probiotics into foods such as snacks, juices and cereal bars can only be done by direct mixing into finished products as such infusion would not involve heating that would inactivate the probiotics. Furthermore, infusion of probiotics in beverages must always be done during the product heat-treatment process prior to the sealing of the beverage packaging to avoid cross-contamination. Consequently, there are limited thermally-processed food and beverage products that include probiotics during processing; the probiotics must be added after the thermal processing, when the food or beverage has cooled down. Such practice is inconvenient or impossible for food such as baked products; and not feasible for pasteurized beverages such as fresh milk or juices that are highly susceptible to bacteria contamination.

Many probiotics exhibit their health benefits when they are alive. Hence, in addition to surviving the manufacturing process and shelf life of food/beverages, probiotics should be also be able to survive the adverse gastro-intestinal tract environment, such as the low pH stomach acid and digestive enzymes, before reaching the small intestine for colonization. Although there are numerous commercial probiotics products, particularly powder/pill form supplements, most of them lose their viability during high temperature food processing, storage, in the gastrointestinal environment. Further, probiotics that are heat-stressed are particularly sensitive to high temperature (Hao et al. 2021; Gardiner et al. 2000) and tend to lose their viability especially when passing through challenging gastrointestinal tract.

Lactobacillus and Bifidobacterium are the most commonly used genera of probiotics with tolerable heat shock below 60° C. (Hao et al. 2021; Gardiner et al. 2000). In order to exert health benefits, a minimum dose of 10⁶ colony-forming unit (cfu) per gram or mL probiotic bacteria is recommended. To address the challenges posed by gastric acid, manufacturers typically add an excessive quantity of probiotics into the product, hoping that a portion would survive both the heat treatment and the gastrointestinal tract. However, this practice is not cost-effective; further, the shelf life of probiotics is uncertain. Additionally, high temperature resistance is still a challenge for manufacturers. Although heat resistant Bacillus has been proposed to be an alternative to Lactobacillus, Bacillus has a spore forming feature that might pose a public health risk due to its toxin and its antibiotic resistance. Moreover, Bacillus are hard to eliminate due to their resistance to industrial disinfection processes. Furthermore, Bacillus does not colonize the gastrointestinal tract well (Bernardeau et al. 2017).

### DESCRIPTION OF THE RELATED ART

Polymers and proteins are the typical ingredients used in formulations to protect probiotics against stomach acid. However, conventional formulations suffer structural changes during high temperature processing and are unable to provide suitable protection against stomach acid without an enteric coating. To date, there are no formulations with a dual protective effect against both thermal stress and gastrointestinal acid.

For example, EP2648528B1 describes a composition and a method for improving stability and extending the shelf life of probiotic bacteria when delivered in a food product, particularly for oxygen and humidity resistance. The multi-layered formulation contains probiotics and a stabilizer/antioxidant as a core, with at least three-layers of coatings comprising hydrophobic solid fat to prevent the penetration of water/humidity into the core, a water-sealed coating as an intermediate coating layer to lower surface tension, an oxygen and humidity sealed coating for reducing transmission of humidity and oxygen to maintain the viability of the probiotics. There is no discussion of protection of the probiotics against high temperature processing.

US 2004/0175389 discloses a formulation for protecting probiotic bacteria during passage through the stomach and release in the intestine. The formulation is in the form of a capsule that includes a water-free mixture of probiotic bacteria with monovalent alginate salts, and an enteric coating. The outer shell of the capsule turns into a gel upon contact with an acidic environment. The gel provides a protective barrier against proton flux into the capsule core. However, this composition is only useful for tablets and capsules under very low water activity storage which requires nitrogen-flushed or vacuum-sealed containers during storage. There is no description relevant to heat protection.

WO 94/00019 describes a method of preparing a baked product containing living microorganisms by injecting live probiotics into the baked product after cooling. However, the injection process is through a multiplicity of needles, which is costly, inconvenient, and may disrupt the texture of the baked product.

EP2451300B1 describes a method of preparing a probiotic granule made up of multiple layers for admixing into healthy food, wherein the microorganisms are stabilized to survive heat processing of the food, particularly dry food. The granule includes probiotics and a substrate where probiotics adsorbed as core, an inner oily layer coating, and two outer layers consist of two different polymers for stabilizing the probiotics in the upper gastrointestinal tract and for heat resistance, respectively. The formulation was exemplified by dry heating at 80° C. for 30 or 45 minutes. No description or direction was provided as of how the layers having protected probiotics against high temperature processing does not affect the acid resistance capability of the heat-treated probiotics. However, the suitability of the granule size to be infused into food and beverage is questionable.

RU2549098C2 describes a method of manufacturing a granule of probiotics containing a probiotic core in a three-layer granule with a first inner oil layer covering the core, a first outer layer to stabilize core in the upper gastrointestinal tract, and second outer layer to stabilize the core under baking conditions. No description or direction was provided regarding how the layers that have protected probiotics against high temperature processing affects the acid resistance capability of the heat-treated probiotics.

CN111134332A discloses a preparation method of probiotic microcapsules by a fluidized bed spray granulation method, for use as a dietary supplement in the food and health care industry. Neither description or direction was provided as of how the layers protect probiotics against high temperature processing or stomach acid.

CN102178238B discloses heat-resistant microencapsulated probiotics with a grain size between 20 and 400 microns subjected to 80 to 90° C. temperature for 3 minutes is reduced by 1 to 1.5 orders of magnitude from 10⁹ to 10¹⁰ cfu/g. No description or direction was provided as of how the encapsulation layers that protect probiotics against high temperature processing affect the acid resistance capability of the heat-treated probiotics.

None of the above compositions provides a mixture that can effectively protect the probiotics in both high temperature processing and against gastrointestinal stomach acid and enzymes challenges for sufficient numbers of live probiotics to reach the intestines. The present invention addresses this problem.

### SUMMARY OF THE INVENTION

A heat and acid resistant probiotics microsphere having a size from 20 to 250 μm that can readily be incorporated into food or beverages that subsequently undergo thermal treatment. The synbiotic core includes a seed layer formed from at least one polysaccharide. A probiotic microorganism is coated on the seed layer. An acid-resistant shell layer is positioned over the synbiotic core, the acid-resistant shell layer comprising one or more pH-responsive polymers. A heat-resistant bilayer shell is positioned over the acid-resistant shell layer, the heat-resistant bilayer shell including an inner shell layer and an outer shell layer, wherein the inner shell layer includes a heat-resistant liposome layer and the outer layer includes a heat-resistant disaccharide or polysaccharide.

In another aspect, the invention relates to a microsphere having a size from 20 to 250 μm, including a granulated synbiotic core. The granulated synbiotic core includes a seed, and the seed has an oligosaccharide and a polysaccharide. The granulated synbiotic core further includes a probiotic microorganism. The granulated synbiotic core may further include one or more binders including a whey protein and a milk protein. The heat and acid resistant probiotics microsphere further includes an acid resistant shell layer, and the acid resistant shell layer has one or more pH-responsive polymers, and the one or more pH-responsive polymers may include Eudragit® L100 and an alginate. The heat and acid resistant probiotics microsphere further includes a heat resistant bilayer shell. The heat resistant bilayer shell has an inner layer and an outer layer, wherein the inner layer includes: one or more isoprenoid-stabilized phospholipid liposomes. The one or more isoprenoid-stabilized phospholipid liposomes have a phospholipid selected from the group consisting of phosphatidylcholine and phosphatidylethanolamine. The inner layer of the heat resistant bilayer shell has one or more isoprenoids. One or more isoprenoids has a cholesterol and a β-carotene. The inner layer of the heat resistant bilayer shell may also include a polysaccharide. The polysaccharide includes a maltodextrin, while the outer layer of the heat resistant bilayer shell has a disaccharide such as trehalose. The outer layer of the heat resistant bilayer shell may further include one or more minerals such as talc.

In another aspect, the heat and acid resistant probiotics particle resists a temperature of 75-90° C. for 5-15 minutes.

In another aspect, the weight percentage of the heat and acid resistant probiotics particle includes 50-79.2% seed, 0.01-0.1% live probiotics, 1.6-3% protein, 2-6.3% polymer, 4-7.9% liposome, 0-1.6% polysaccharides, 0-3.2% disaccharides, and 0-0.3% minerals.

In another aspect, the weight percentage of the heat and acid resistant probiotics particle includes 79.2% seed, 0.01% live probiotics, 1.6% protein, 6.3% polymer, 7.9% liposome, 1.6% polysaccharides, 3.2% disaccharides, and 0.3% minerals.

In another aspect, the acid resistant shell layer and the heat resistant bilayer shell have at least four layers.

In another aspect, the heat and acid resistant probiotics particle maintains viability of more than 6 Log CFU/g after gastrointestinal tract challenges.

In another aspect, the heat and acid resistant probiotics particle has an additional water barrier coating layer coupled to the outer layer.

In another aspect, the high temperature resistant probiotics particle resists temperature at 90° C. for 15 minutes.

In another aspect, the additional water barrier coating layer has 6-10% shellac and dimethylaminoethyl methacrylate-copolymer Eudragit® EPO.

In another aspect, the heat and acid resistant probiotics particle is infused into liquid beverages that are subjected to thermal treatment such as milk and juice prior to pasteurization.

The present invention also provides a method for preparing a probiotics particle resistant to high temperature and gastrointestinal digestive challenges, the method comprising:
a. Preparing a seed with size between 10-125 μm and density between 0.65 to 0.75 g/cm³ by sieving;
b. Preparing a homogenous live probiotics solution with prebiotics and proteins to enhance probiotics viability and granulation by stir-mixing;
c. Preparing an acid resistant pH-responsive polymer by heating and stir-mixing;
d. Preparing an isoprenoid-stabilized phospholipid liposome by stir-mixing of proliposome that are prepared using a thin-film method;
e. Preparing a heat resistant outer layer comprising a disaccharide by stir-mixing;
f. Preparing the high temperature resistant probiotics microsphere with the seed coated with a prebiotic and a probiotic layer, followed by acid resistant layer and heat resistant bilayers by fluidized-bed coating.

In another aspect, the method for preparing the probiotics particle further includes a seed core, a probiotic with binder coating on seed surface, an acid resistant protective layer, and a plurality of heat resistant protective layers.

In another aspect, the method for preparing the probiotics particle further includes an encapsulation efficiency of microsphere more than 95%.

In another aspect, the method for preparing the probiotics particle further includes a viable count of 10⁸-10¹⁰ CFU/g.

In another aspect, the probiotics particle has a size in the range of 20-250 μm, with a means size of 167 μm.

In another aspect, the method for preparing the probiotics particle further includes infusing the probiotics particle into a food product, a food additive, or liquid beverages such as milk or juice prior to pasteurization.

In another aspect, the food product in which the probiotics particle is infused further comprises dry food that is subjected to thermal treatment such as a full baking production process such as pastry and bread.

### BRIEF DESCRIPTION OF THE DRAWINGS

FIG. 1 shows a predicted structure of heat resistant core-shells system for probiotics fabricated using fluidized bed system.

FIG. 2 shows a viability of high temperature resistant probiotics before and after simulated gastric acid challenge and intestinal released tests.

### DETAILED DESCRIPTION

The present invention solves the problem of viability loss when heat sensitive nutrients/probiotics directly infuse into food product, by providing a core-shell encapsulation system that can withstand high temperature for a period of time. Additionally, the core-shell system provides protection to the probiotics such that a sufficient amount of viable probiotic is delivered to the host and can further survive subsequent gastrointestinal digestive challenges. The high temperature resistant probiotics involve specialized encapsulation formulation which is feasible for large scale manufacturing using fluidized-bed coating. Coating with a fluidized-bed is both economical and convenient as both the coating and drying processes can be considered as a single process with the same equipment. Dried powder product can be obtained through a one-step fabrication process in the fluidized-bed within few hours having a desirable micro-sized particle. The resulting minute size of the formed particles facilitates the direct infusion of probiotics into food and beverage for easy the delivery of probiotics to host.

Development of a Heat and Acid Resistant Probiotics Particle 100

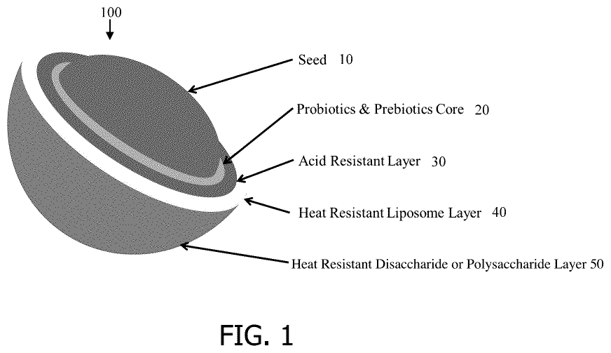

FIG. 1 depicts an example of a heat and acid resistant probiotics particle 100 according to an embodiment. The particle may take the form of a microsphere in one aspect of the present invention. The heat and acid resistant probiotics microsphere 100 is made up of a seed 10 and four layers with specific functions: probiotics and prebiotics core layer 20, acid resistant layer 30, heat resistant liposome layer 40, and heat resistant disaccharide or polysaccharide layer 50.

The core-shell particle 100 includes a seed 10. Seed 10 may be selected from materials that can provide nutrition to the probiotic. Exemplary materials may be one or more of: sucrose sphere, inulin, starch, cellulose. Selection of seed material is made based on the material's coatability. In particular different wetting properties impact the deposition rate on the seed particles. Poor wetting abilities result in poor coating efficiency, leading to viability loss (Example 2, Table 2). Typical seed/core sizes are between 10-125 μm and have a density between 0.65 to 0.75 g/cm³.

Positioned over the seed 10 is a probiotics and prebiotics core layer 20 that may be one or more probiotics such as Bifidobacterium, Lactobacillus, Lactococcus, Leuconostoc, Streptococcus, Enterococcus, Staphylococcus, Saccharomyces and Kluyveromyces. The probiotics and prebiotics core layer 20 may further include prebiotics for providing nutrition to the probiotics. The prebiotics may be selected from one or more of fructo-oligosaccharides, galacto-oligosacchrides, inulin, pectin, proteins such as whey protein, soy protein, chickpea protein, rice protein, pea protein, egg protein, casein, milk protein, zein and bovine serum albumin as a binder to agglomerate and encapsulate the prebiotics so as to form free-flowing particles. In addition to probiotics and optional prebiotics, heat sensitive nutrients may also be provided in the probiotics and prebiotics core layer 20. These include vitamin such as vitamin C, vitamin B1, and vitamin E.

An acid-resistant layer 30 is formed over and encapsulating the probiotics and prebiotics core layer 20. The acid-resistant encapsulation layer 30 that covers the probiotics and prebiotics core layer 20 may be a pH-responsive polymer such as Eudragit® L100, Eudragit®L100-55, Acryl-EZE®, shellac, alginate, pectin or mixtures of these materials. This layer provides a controlled release function within the small intestine following ingestion, surviving dissolution by stomach acid. The selected polymer with pH-dependent solubility will gel at a pH below 3 but dissolve rapidly upon deprotonation of carboxylic acid groups at pH >6.

Heat resistant bilayer comprises an inner liposome layer 40 and an outer disaccharide or polysaccharide layer 50. Heat resistant liposome layer 40 may be a proliposome that is more stable than liposome, easy to distribute, transfer and store. Liposomes made from proliposome with components comprising phospholipids may be selected from one or more of phosphatidylcholine, phosphatidylethanolamine, phosphatidylinositol, dipalmitoyl phosphatidylcholine, dipalmitoyl phosphatidylethanolamine, dipalmitoyl phosphatidylinositol, dimyristoyl phosphatidylcholine, dimyristoyl phosphatidylethanolamine, dimyristoyl phosphatidylinositol; isoprenoids compounds can be selected from cholesterol, β-carotene and lycopene; water soluble carrier can be selected from maltodextrin, sorbitol, mannitol, maltitol, xylitol, or any combinations thereof.

The fabrication of proliposomes includes mixing a ratio of ingredients which are phospholipid (phosphatidylcholine, phosphatidylethanolamine, phosphatidylinositol, dipalmitoyl phosphatidylcholine, dipalmitoyl phosphatidylethanolamine, dipalmitoyl phosphatidylinositol, dimyristoyl phosphatidylcholine, dimyristoyl phosphatidylethanolamine and dimyristoyl phosphatidylinositol), isoprenoids compounds (cholesterol, β-carotene and lycopene) and water soluble carriers (maltodextrin, sorbitol, mannitol, maltitol, xylitol). Isoprenoids (cholesterol and beta-carotene) can stabilize the phospholipid bilayer by a nail- and rivet-like mechanism. They stabilize phospholipid bilayer by inserting themselves within the phospholipid, forming a hydrogen bond with the head group of phospholipids; and allow van der Waals attractive forces to align the adjacent lipid chains in order. Their ability to stabilize the structure and the resulting heat resistance capability in the development of heat resistant bilayer were studied by heating tests. A water-soluble carrier (maltodextrin, sorbitol, mannitol, maltitol, xylitol) may be used to support the phospholipids during the preparation of proliposomes due to their high surface area and porosity, allowing rapid conversion of liposomal dispersion on hydration.

A thin-film method may be used to prepare an isoprenoid-stabilized phospholipid liposome. Phospholipid, isoprenoid and water-soluble carrier may be dissolved in a solvent, such as ethanol, followed by placing solvent removal to obtain a dry granular thin-film material using a rotary evaporation system. The powder-form proliposome is then re-dissolved in water to form liposomes prior to use. The liposome structure provides a heat-resistant property. Spray coating of an ethanolic solution containing similar compositions of phospholipid and isoprenoids may not confer the desirable heat resistant characteristic to the probiotics (Example 5, Table 10).

The outer heat resistant layer 50 includes a disaccharide or polysaccharide with an optional mineral addition. Disaccharides may be one or more of sucrose, lactose, maltose, trehalose, cellobiose, or chitobiose. Talc, kaolin, ZnO, SiO₂ and SiO₂, or combinations thereof may be used as the mineral addition. An exemplary disaccharide is trehalose with an addition of approximately 1% talc to increase the flowability of the fluidized powder due to the high viscosity of the disaccharide.

The heat resistant property of bilayer 40/50 absorbs kinetic energy during heating, enabling the phospholipid molecules to stay further apart and create a gap space among the phosphate head and acyl lipid chains which is unfavorable to heat transfer. Insertion of isoprenoids within the phospholipid by nail- and rivet-like mechanism assist in stabilizing the bilayer structure by forming hydrogen bonds with the head group of phospholipids while allowing van der Waals attractive forces to align the adjacent lipid chains in order. The outer layer 50, for example, trehalose, which reduces heat transfer due to the highly stable bonding of the two hexoses and the strong intermolecular hydrogen bonds that bind the trehalose tightly into a giant cluster rigid with low molecular mobility.

Exemplary ingredient % weight in the particles of the present invention are as follows: a granulated synbiotic core of 50-79.2% seed; 0.01-0.1% live probiotics materials, 1.6-3% protein and 0-1.6% polysaccharides; b) the acid resistant shell layer of 2-6.3% polymer; and c) the heat resistant bilayer shell of inner layer 4-7.9% liposome, and outer layer 0-3.2% disaccharides, and 0-0.3% minerals.

Spray granulation and fluidized-bed coating are methods to form granules and forming coatings on granules or particles, respectively. Spray granulation is used to first produce the synbiotic core where a liquid mixture containing prebiotics, probiotics and binders are dried simultaneously while forming dust free granules. In fluidized-bed coating, the synbiotic core powder is fluidized by flowing vertical air through a distributor plate at the bottom of the system. The coating material, which can be a melt, a suspension or a solution dissolved in water or organic solvent, is sprayed onto the fluidized particles, where the single droplets collide with the particle surface and spread, whereas the solvent evaporates constantly. The remaining solid component deposits on the particle surface and forms a shell of layers, causing growth of the particle. The aim of this process is the homogeneous deposition of droplets on a single particle and over an entire particle population, which is critical to achieve a homogeneous and smooth-out coating layer.

The high temperature resistant probiotics are infused foods and beverages, for example, a dough to be baked or a liquid to be pasteurized. The outer bilayer shell offers heat shielding during food processing while the acid-resistant layer overcomes the challenges of the gastrointestinal tract achieve a controlled release when reaching small intestine to deliver sufficient live probiotics.

### Example 1

Experimental Set-Up for Heating Test and Viability Testing

Heating test was conducted using a convection oven. A digital oven thermometer with its probe was kept inside the oven to detect the temperature. It was connected with a steel cable to the display that was kept outside of oven for monitoring. As heat escape after the opening of oven's door when samples were inserted, the 15 mins heating time was started only when the temperature display showed 90° C. After heating, samples were cooled down for 30 mins in the biosafety cabinet. After that, the encapsulated probiotics were released into formulated releasing solutions containing bile salt, pancreatin and lipase suspended in sodium bicarbonate at pH 6.8 using a paddle homogenizer with 50 rpm paddle movement at 37° C. for 30 mins. The mixture was seeded on MRS agar and incubated at 37° C. for 24-48 hours. Probiotics colonies formed on the surface of the agar plate. The amount of cfu/g of sample was quantified as the viable probiotics.

### Example 2

Heat resistant performance of high temperature resistant probiotics wherein shells layer comprises specialized filamentous polymers attached with cholesterol-stabilized phosphatidylcholine liposome cum trehalose bilayer

Probiotics and Prebiotics Core Development

During the selection of seed for synbiotic core development, particle with different wetting properties would have highly affected the deposition rate on particles and caused poor coating efficiency, leading to viability loss. Thus, in trial A (Table 1) using freeze-dried probiotics as seed, the viability loss after heating test was comparable to the control (Table 2). Then, reformulation was done by using inulin as the carrier, while the core layer comprising a mixture of probiotics and prebiotics with whey protein as a binder to agglomerate and encapsulate the inulin carrier to form free-flowing particles.

TABLE 1
Formulation of seed and core layer for Trial A and high temperature
resistant probiotics in the synbiotic core development

| | | High temperature resistant |
|---|---|---|
| | Trial A | probiotics |
| Seed (250 g) | Freeze-dried Probiotics | Inulin |
| | (Lactobacillus | |
| | delbrueckii subsp. | |
| | bulgaricus) | |
| | Prebiotics: Fructo- | |
| | oligosaccharides (FOS) | |
| | Ratio (Probiotics:prebiotics) = | |
| Core layer | 5:1 w/w | Mixture of 100 mL water |
| | | containing: |
| | | 9.98 × 10¹¹ CFU |
| | | Probiotics: Lactobacillus |
| | | delbrueckii subsp. |
| | | bulgaricus |
| | | 5% (w/w) Prebiotics: |
| | | Fructo-oligosaccharides |
| | | (FOS) |
| | | 5% (w/w) Whey protein: |
| | | binder |

TABLE 2
Viability and log reduction of probiotics before and after heating

| | Before heating | After heating | |
|---|---|---|---|
| | (Log CFU/g) | (Log CFU/g) | Log reduction |
| Control (freeze-dried | 9.58 ± 0.01 | 7.79 ± 0.31 | 1.79 |
| probiotics only) | | | |
| Trial A | 8.38 ± 0.03 | 6.72 ± 0.43 | 1.67 |
| Control (inulin | 7.83 ± 0.03 | 6.99 ± 0.12 | 0.83 |
| coated with | | | |
| probiotics) | | | |
| High temperature | 8.96 ± 0.01 | 8.80 ± 0.14 | 0.16 |
| resistant probiotics | | | |

Inulin (10-250 μm, 0.70 g/cm³) was used as the seed. The core layer comprises mixture of probiotics and prebiotics with whey protein as a binder to agglomerate and coat the inulin seed to form free-flowing particles. Pre-cultured probiotics up to 10¹¹ CFU/g was washed and mixed with 50% w/w prebiotics and 50% w/w whey protein, before spray coating on inulin. Fluidized bed parameters are having flow rate of 20 m³/h, product temperature of 41.3° C., spray rate of 2.0 g/min and spray pressure of 0.4 bar. A 500 weight gain is designed for this purpose.

Acid Resistant Layer 30 Development

The acid resistant layer 30 is for controlled release function at small intestinal tract upon consumption of high temperature resistant probiotics. Eudragit® L100 is a polymer with pH-dependent solubility that dissolve rapidly upon deprotonation of carboxylic acid groups at pH >6.

Formulation with acid resistant layer 30 without pretreatment (preheated) affected the heat resistant competency of the heat resistant shells after subjecting to heating in a convection oven at 90° C. for 15 min (Table 3). The encapsulated probiotics suffered huge loss where the viability after heating dropped below 6 Log cfu/g.

TABLE 3
Viability and log reduction of encapsulated probiotics
before and after heating test with direct coated
acid resistant layer 30 (without pre-warmed)

| | Before heating | After heating | Log reduction |
|---|---|---|---|
| | (Log CFU/g) | (Log CFU/g) | (Log CFU/g) |
| Control | 10.03 | 6.53 | 3.50 |
| Encapsulated | 8.72 | 5.94 | 2.77 |
| probiotics | | | |
| formulation | | | |

*Control powder: Inulin coated with probiotics

Pre-warming, spraying temperature and solute concentrations affect its filament formation. 20% w/w of Eudragit® L100 was pre-warmed at 50° C. for 12 hr prior spraying at flow rate of 25 m³/h, product temperature of 41° C., spray rate of 3.2 g/min and spray pressure of 0.4 bar. The treatment condition enabled the adhesion of heat resistant liposome layer without deteriorating the heat resistant capability; while maintain the novel function of its pH-responsiveness. An 8% weight gain is designed for this purpose.

Heat Resistant Bilayer Development

Heat resistant bilayer comprises an inner isoprenoid-stabilized phospholipid liposome layer 40 and an outer trehalose layer 50. Proliposome for the formation of isoprenoid-stabilized phospholipid liposome after encapsulation was first prepared. Proliposome is an aqueous-soluble dry phospholipid particle that is easy to distribute, transfer and store, and this is beneficial to future scale-up production. Liposomes can be formed from proliposome upon addition of aqueous solvent and shaking. The fabrication of proliposome includes mixing the optimum ratio of four ingredients which are phospholipid (phosphatidylcholine (PC)), isoprenoids (cholesterol or beta-carotene) and water-soluble carriers (maltodextrin and ethanol). Briefly, PC, isoprenoid and maltodextrin were mixed in ethanol, followed by placing them in a round-bottom flask to remove the ethanol to obtain a dry granular material using a rotary evaporation system. This powder formed proliposome that was then re-dissolved in water to form liposome right before use. Optimum ratio of isoprenoid-stabilized phospholipid liposome (soluble in ethanol & can be re-dissolved in water) consisted of 4 g PC, 0.8 g isoprenoid and 20 g maltodextrin in 160 g water. Fluidized bed parameter has flow rate of 30 m³/h, product temperature of 43.5° C., spray rate of 2.0 g/min and spray pressure of 0.4 bar. A 9% weight gain is designed for this purpose.

The outer trehalose layer is spray coated at 10%, with additional 1% of talc to reduce powder stickiness during fluidized bed coating. Fluidized bed parameters are having flow rate of 30 m³/h, product temperature of 40.5° C., spray rate of 2.0 g/min and spray pressure of 0.4 bar. A 3.6% weight gain is designed for this purpose.

TABLE 4
Formulation of heat resistant bilayers in
high temperature resistant probiotics

| Seed | Inulin |
|---|---|
| Core layer | Mixture of 100 ml solution containing: |
| | Probiotics Lactobacillus delbrueckii subsp. bulgaricus |
| | Prebiotics: fructooligosaccharides |
| | Binder: whey protein |
| pH- | 200 g Ethanolic solution containing: |
| responsive | Eudragit ® L100 |
| layer | |
| Inner heat | Mixture of 160 g liposome solution containing: |
| resistant layer | Phosphotidylcholine (PC) |
| | Cholesterol |
| | Maltodextrin |
| Outer heat | Mixture of 100 g solution containing: |
| resistant layer | Trehalose |
| | Talc |

The encapsulated probiotics only loss 0.16 Log CFU/g from 8.96 Log CFU/g viability, compared to 0.83 Log CFU/g in control (from 7.83 Log CFU/g) after heating in a convection oven at 90° C. for 15 min (Table 5).

TABLE 5
Viability and log reduction of high temperature resistant
probiotics before and after heating test

| | Before heating | After heating | Log reduction |
|---|---|---|---|
| | (Log CFU/g) | (Log CFU/g) | (Log CFU/g) |
| Control | 7.83 ± 0.03 | 6.99 ± 0.12 | 0.83 |
| High temperature | 8.96 ± 0.01 | 8.80 ± 0.14 | 0.16 |
| resistant probiotics | | | |

*Control: Inulin coated with probiotics

### Example 3

Heat resistant performance different concentrations of β-carotene-stabilized phosphatidylcholine liposome as heat resistant layer of high temperature resistant probiotics

Probiotics and Prebiotics Core Development

Inulin was used as seed. The core layer comprising mixture of probiotics and prebiotics with whey protein as a binder to agglomerate and coat the inulin seed to form free-flowing particles. Pre-cultured probiotics up to 101 CFU/g was washed and mixed with 5% w/w prebiotics and 5% w/w whey protein, before spray coating on inulin. Fluidized bed parameters comprises: flow rate 20 m³/h, product temperature 41.3° C., spray rate 2.0 g/min and spray pressure of 0.4 bar. A 5% weight gain is designed for this purpose.

Acid Resistant Layer 30 Development

Pre-warming, spraying temperature and solute concentrations affect its filament formation. 20% w/w of Eudragit® L100 was pre-warmed at 50° C. for 12 hr prior spraying at flow rate of 25 m³/h, product temperature of 41° C., spray rate of 3.2 g/min and spray pressure of 0.4 bar. The treatment condition assisted the adhesion of heat resistant liposome layer without deteriorating the heat resistant capability, and it maintains the novel function of its pH-responsiveness. An 8% weight gain is designed for this purpose.

Heat Resistant Liposome Layer Development

Proliposome powder consisting of phosphatidylcholine, beta-carotene and maltodextrin was re-dissolved in water to form liposome right before use. Ratio of isoprenoid-stabilized phospholipid liposome (LP-BCA) was 0.5× or 1× (4 g PC, 0.8 g beta-carotene and 20 g maltodextrin in 160 g water). Fluidized bed parameter has a flow rate of 30 m³/h, product temperature of 43.5° C., spray rate of 2.0 g/min and spray pressure of 0.4 bar. A 9% weight gain is designed for this purpose.

TABLE 6
Formulation of heat resistant bilayers in
high temperature resistant probiotics

| Seed | Inulin |
|---|---|
| Core layer | Mixture of 100 ml solution containing: |
| | Probiotics Lactobacillus delbrueckii subsp. bulgaricus |
| | Prebiotics: fructooligosaccharides |
| | Binder: whey protein |
| pH- | 200 g Ethanolic solution containing: |
| responsive | Eudragit ® L100 |
| layer | |
| Inner heat | Mixture of liposome solution containing: |
| resistant layer | Phosphotidylcholine (PC) |
| | β-carotene |
| | Maltodextrin |

Heat resistant capability of probiotics after heating test was better for heat resistant layer formulated with 0.5× liposome (0.72 Log CFU/g viability loss) compared to 1× liposome (0.97 Log CFU/g viability loss). Control has 0.95 Log CFU/g viability loss after heating in a convection oven at 90° C. for 15 min (Table 7).

TABLE 7
Viability and log reduction of high temperature resistant probiotics
fabricated using formulation in Table 6 before and after heating test

| | Before heating | After heating | Log reduction |
|---|---|---|---|
| | (Log CFU/g) | (Log CFU/g) | (Log CFU/g) |
| Control | 8.22 ± 0.01 | 7.27 ± 0.03 | 0.95 |
| High temperature | 8.99 ± 0.03 | 8.27 ± 0.06 | 0.72 |
| resistant probiotics | | | |
| with 0.5x LP-BCA | | | |
| High temperature | 9.01 ± 0.03 | 8.05 ± 0.06 | 0.97 |
| resistant probiotics | | | |
| with 1x LP-BCA | | | |

*Control: Inulin coated with probiotics

### Example 4

Heat Resistant Performance of Different Concentrations of Cholesterol-Stabilized Phosphatidylcholine Liposome as Heat Resistant Layer of High Temperature Resistant Probiotics

Probiotics and Prebiotics Core 20 Development

Using inulin as seed, the core layer comprises a mixture of probiotics and prebiotics with whey protein as a binder to agglomerate and coat on the inulin seed to form free-flowing particles. Pre-cultured probiotics up to 10¹¹ CFU/g was washed and mixed with 5% w/w prebiotics and 5% w/w whey protein, before spray coating on inulin. Fluidized bed parameter comprises: flow rate 20 m³/h, product temperature 41.3° C., spray rate 2.0 g/min and spray pressure of 0.4 bar. A 5% weight gain is designed for this purpose.

Acid Resistant Layer 30 Development

Pre-warming, spraying temperature and solute concentrations affect its filament formation. 20% w/w of Eudragit® L100 was pre-warmed at 50° C. for 12 hr prior spraying at flow rate of 25 m³/h, product temperature of 41° C., spray rate of 3.2 g/min and spray pressure of 0.4 bar. The treatment condition assisted the adhesion of heat resistant liposome layer without deteriorating the heat resistant capability; while maintain the novel function of its pH-responsiveness. An 8% weight gain is designed for this purpose.

Heat Resistant Liposome Layer Development

Proliposome powder consisting of phosphatidylcholine, beta-carotene and maltodextrin was re-dissolved in water to form liposome right before use. Ratio of isoprenoid-stabilized phospholipid liposome (LP-CHL) was 1× or 2× (4 g PC, 0.8 g cholesterol and 20 g maltodextrin in 160 g water). Fluidized bed parameters are having flow rate of 30 m3/h, product temperature of 43.5° C., spray rate of 2.0 g/min and spray pressure of 0.4 bar. A 9% weight gain is designed for this purpose.

TABLE 8
Formulation of heat resistant bilayers in
high temperature resistant probiotics

| Seed | Inulin |
|---|---|
| Core layer | Mixture of 100 ml solution containing: |
| | Probiotics Lactobacillus delbrueckii subsp. bulgaricus |
| | Prebiotics: fructooligosaccharides |
| | Binder: whey protein |
| pH- | 200 g Ethanolic solution containing: |
| responsive | Eudragit ® L100 |
| layer | |
| Inner heat | Mixture of liposome solution containing: |
| resistant layer | Phosphotidylcholine (PC) |
| | Cholesterol |
| | Maltodextrin |

Heat resistant capability of formulations after heating test was better for heat resistant layer formulated with 1× liposome (0.21 Log CFU/g viability loss of probiotics) compared to 2× liposome (0.48 Log CFU/g viability loss of probiotics). Control has 0.59 Log CFU/g viability loss after heating in a convection oven at 90° C. for 15 min (Table 9).

TABLE 9
Viability and log reduction of high temperature resistant probiotics
fabricated using formulation in Table 8 before and after heating test

| | Before heating | After heating | Log reduction |
|---|---|---|---|
| | (Log CFU/g) | (Log CFU/g) | (Log CFU/g) |
| Control | 8.46 ± 0.18 | 7.97 ± 0.17 | 0.59 |
| High temperature | 8.97 ± 0.11 | 8.75 ± 0.06 | 0.21 |
| resistant probiotics | | | |
| with 1x LP-CHL | | | |
| High temperature | 8.95 ± 0.08 | 8.46 ± 0.07 | 0.48 |
| resistant probiotics | | | |
| with 2x LP-CHL | | | |

*Control: Inulin coated with probiotics

Heat resistant capability for formulation coated with ethanolic mixture of phospholipid and isoprenoid (200 g of 3% PC+1.5% cholesterol+1.5% ATO5+4% Talc; instead of liposome) was unsatisfactory. The formulation also involved acid resistant coating (Eudragit® L100) that was not pre-heated. Results showed that the encapsulated probiotics suffered higher loss in viability than the unformulated control after heating test (2.7 Log CFU/g for encapsulated probiotics vs 1.7 Log CFU/g for unformulated control).

TABLE 10
Viability and log reduction of encapsulated probiotics coated
with ethanolic mixture of phospholipid and isoprenoid

| | Before heating | After heating | Log reduction |
|---|---|---|---|
| | (Log CFU/g) | (Log CFU/g) | (Log CFU/g) |
| Control | 9.72 | 8.03 | 1.69 |
| Encapsulated | 10.16 | 7.42 | 2.74 |
| Probiotics | | | |

*Control: Inulin coated with probiotics

### Example 5

Heat Resistant Performance of Cholesterol-Stabilized Phosphatidylcholine Cum Different Concentrations of Trehalose as Heat Resistant Bilayer of High Temperature Resistant Probiotics

Probiotics and Prebiotics Core 20 Development

Inulin was selected as seed. The core layer comprises mixture of probiotics and prebiotics with whey protein as a binder to agglomerate and coat the inulin seed to form free-flowing particles. Pre-cultured probiotics up to 10¹¹ CFU/g was washed and mixed with 5% w/w prebiotics and 5% w/w whey protein, before spray coating on inulin. Fluidized bed parameter comprises: flow rate 20 m³/h, product temperature 41.3° C., spray rate 2.0 g/min and spray pressure of 0.4 bar. A 5% weight gain is designed for this purpose.

Acid Resistant Layer 30 Development

Pre-warming, spraying temperature and solute concentrations affect its filament formation. 20% w/w of Eudragit® L100 was pre-warmed at 50° C. for 12 hr prior spraying at flow rate of 25 m³/h, product temperature of 41° C., spray rate of 3.2 g/min and spray pressure of 0.4 bar. The treatment condition assisted the adhesion of heat resistant liposome layer without deteriorating the heat resistant capability; while maintain the novel function of its pH-responsiveness. An 8% weight gain is designed for this purpose.

Heat Resistant Bilayer Development

Proliposome powder consisting of phosphatidylcholine, beta-carotene and maltodextrin was re-dissolved in water to form liposome right before use. Ratio of isoprenoid-stabilized phospholipid liposome (LP-CHL) was 1× or 2× (4 g PC, 0.8 g cholesterol and 20 g maltodextrin in 160 g water). Fluidized bed parameter has flow rate of 30 m³/h, product temperature of 43.5° C., spray rate of 2.0 g/min and spray pressure of 0.4 bar. A 9% weight gain is designed for this purpose.

The outer trehalose layer is spray coated at 5% and 10%, with additional 1% of talc to reduce powder stickiness during fluidized bed coating. Fluidized bed parameter has flow rate of 30 m³/h, product temperature of 40.5° C., spray rate of 2.0 g/min and spray pressure of 0.4 bar. A 3.60 weight gain is designed for this purpose.

TABLE 11
Formulation of heat resistant bilayers in
high temperature resistant probiotics

| Seed | Inulin |
|---|---|
| Core layer | Mixture of 100 ml solution containing: |
| | Probiotics Lactobacillus delbrueckii subsp. bulgaricus |
| | Prebiotics: fructooligosaccharides |
| | Binder: whey protein |
| pH- | 200 g Ethanolic solution containing: |
| responsive | Eudragit ® L100 |
| layer | |
| Inner heat | Mixture of liposome solution containing: |
| resistant layer | Phosphotidylcholine (PC) |
| | Cholesterol |
| | Maltodextrin |
| Outer heat | Mixture of 100 g solution containing: |
| resistant layer | Trehalose |
| | Talc |

Lower concentration of trehalose (500 w/w=5 g) showed greater heat resistant capability (0.16 Log CFU/g loss in viability) than 1000 w/w (0.27 Log CFU/g loss in viability). Viability loss of the control (0.83 Log CFU/g) was 5-fold and 3-fold higher than the respective trehalose's trial.

TABLE 12
Viability and log reduction of high temperature
resistant probiotics fabricated using formulation
in Table 11 before and after heating test

| | Before heating | After heating | Log reduction |
|---|---|---|---|
| | (Log CFU/g) | (Log CFU/g) | (Log CFU/g) |
| Control | 7.83 ± 0.03 | 6.99 ± 0.12 | 0.83 |
| Trehalose 5% | 8.96 ± 0.01 | 8.80 ± 0.14 | 0.16 |
| Trehalose 10% | 8.98 ± 0.01 | 8.71 ± 0.03 | 0.27 |

*Control: Inulin coated with probiotics

### Example 6

Encapsulation Efficiency of High Temperature Resistant Probiotics

The encapsulation efficiency is the survival rate of the probiotics after the encapsulation process that is crucial in delivering live probiotics by the as developed core-shells system. Probiotics viability were evaluated by plate count method and encapsulation efficiency were calculated by comparing the viable probiotics entrapped with the initial number of probiotics added.

TABLE 13
Viability and log reduction of high temperature resistant
probiotics after the encapsulation process

| Total added number of free | 9.98 Log CFU/g |
|---|---|
| cells L. bulgaricus | |
| Total released number of viable | 9.57 Log CFU/g |
| entrapped cells L. bulgaricus in | |
| the final product | |
| Encapsulation efficiency | 97.72% |

Encapsulation efficiency=log 10(released number of viable entrapped cells)/log 10 (added number of free cells)×100.

### Example 7

Particle Size Distribution (Volume-Based) of High Temperature Resistant Probiotics

The particle size of high temperature resistant probiotics was measured using Dynamic Image Analysis Camsizer X2 (wet module) in 100% ethanol. The equivalent spherical diameter, d50 of the powder is 161.77 μm.

TABLE 14
Volume-based particle size distribution and its characteristic
diameters of high temperature resistant probiotics

| | Mean Diameter (μM) |
|---|---|
| d₁₀ | 41.26 |
| d₅₀ | 161.77 |
| d₉₀ | 235.10 |

### Example 8 Heat and Acid Treatment

Simulated Gastrointestinal Challenges and Release of High Temperature Resistant Probiotics

Simulated gastric acid resistant property of heat-treated high temperature resistant probiotics was conducted by performing simulated gastric acid resistant test assay. Heat-treated high temperature resistant probiotics was incubated in simulated gastric juice (pepsin, pH 3) at 37° C. for 2 hours with continuous shaking at 120 rpm. After incubation, the viability of probiotics was evaluated by quantifying the number of viable probiotics before and after incubation in simulated gastric condition using spread-plate method.

Release of heat-treated high temperature resistant probiotics from simulated small intestinal release test was conducted following the gastric acid test assay, where they were incubated into simulated small intestine environment at bile containing pH 6.8 for another 2 hours at 37° C. with continuous shaking at 120 rpm. After incubation, the viability of probiotics was evaluated by quantifying the amount of viable probiotic before and after incubation in simulated gastric and intestinal conditions using spread-plate method.

Acid resistant layers of high temperature resistant probiotics was able to protect the encapsulated probiotics where the released probiotics after simulated gastric acid challenge at pH 3, 37° C. for 2 hours were 0.70 Log CFU/g, compared to control—1.60 Log CFU/g.

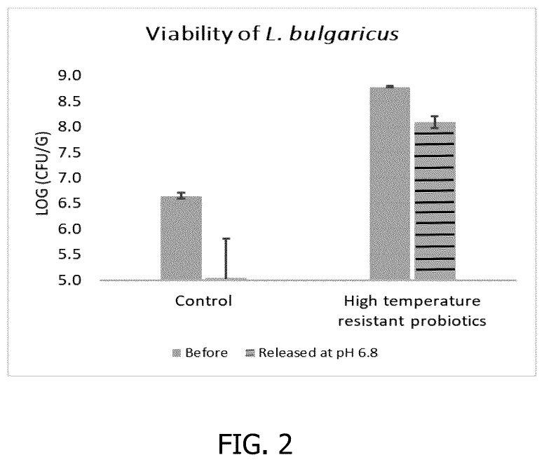

Furthermore, the acid-challenged high temperature resistant probiotics (8.79±0.01 Log CFU/g) was released efficiently from the encapsulation materials in the simulated small intestinal conditions, where it achieved 92% released (8.09±0.12 Log CFU/g—more than 6 Log CFU/g) of probiotics were detected. On the other hand, control did not meet the minimum effective dose after simulated gastric acid challenge and small intestinal release tests (5.05±0.75 Log CFU/g) (FIG. 2).

### Example 9

Heat Resistant Assessment of High Temperature Probiotics According to Present Invention in a Full Bread Baking Condition

Buns and rolls are among baked goods that prevailed during the pandemic. They are the irreplaceable staple breakfast by many consumers all over the world as appetizingly fresh and convenience. Thus, whether the infusion of probiotics into bread/baked products could bring the benefits to the consumers and thus, worth investigation. The stability of the encapsulated probiotics in a bread baking process used in the industry was evaluated.

TABLE 15
Formulation of heat resistant bilayers in
high temperature resistant probiotics

| Seed | Inulin |
|---|---|
| Core layer | Mixture of 100 ml solution containing: |
| | Probiotics Lactobacillus delbrueckii subsp. bulgaricus |
| | Prebiotics: fructooligosaccharides |
| | Binder: whey protein |
| pH- | 200 g Ethanolic solution containing: |
| responsive | Eudragit ® L100 |
| layer | |
| Inner heat | Mixture of 160 g liposome solution containing: |
| resistant layer | Phosphatidylcholine (PC) |
| | Cholesterol |
| | Maltodextrin |
| Outer heat | Mixture of 100 g solution containing: |
| resistant layer | Trehalose |
| | Talc |

Firstly, 9 g of dry yeast was mixed with 4 g of sugar and 80 mL of warm water (≤40° C.) in order to activate the yeast. The mixture was sat at the side around 15 mins until bubbly. Then, 500 g of strong flour, 70 g of sugar, 3 g of salt, 60 g of egg, 200 mL of water and the bubbly yeast mixture were all added into a large bowl and stirred until the dough have a rough surface. It was then rested for 30 min. 30 g of unmelted butter was then added into the dough. The dough was then placed into a mixer (Bosch) and kneaded until the surface of the dough becoming smooth and not sticky. The dough was then transferred into a clean large bowl and covered with cling film and put at a warm spot. The dough was rested for about 1 hour or until double in size. After that, the fillings of bun comprising 2 g (about 9.24 Log cfu/g) of probiotics powder (Formulation in Table 15)-2 g coconut oil mixture was added into the center of 60 g dough that was shaped into a bun. The formed dough was rested in oven at 30˜40° C. for 1 hr or until them double in size. Then, the bun was baked in a pre-heated oven at 180° C. on the middle shelf for 15 minutes. The internal temperature of the bun was monitored that the core of the bun reached 90° C. in 8 minutes and the temperature continued to rise till 98° C. for the remaining 7 minutes. The buns were removed immediately from the oven after baking and left to cool on a wire rack. Viable counts of probiotics in the bun were determined before and after baking. The encapsulated probiotics was released into the formulated releasing solutions at pH7.4 at 37° C., 120 rpm for 2 hours. The mixture was serially diluted and seeded on MRS agar and incubated at 37° C. for 24-48 hours. Probiotics colonies formed on the surface of the agar plate was calculated as viable probiotics. Probiotics without encapsulation was used as the control.

Upon baking, all the probiotics was inactivated in bun made with control. For samples containing high temperature resistant probiotics, a slight decrease in probiotics viability where the bun contained 8.39 Log CFU of live probiotics. This finding indicate that the coating layers of the invention provide the probiotics with heat resistance that exerted during baking when used as the fillings in bun.

TABLE 16
Viable counts of probiotics in buns before and after
baking of buns (60 g) at 180° C. for 15 mins

| | Before baking | After baking |
|---|---|---|
| | (Log CFU) | (Log CFU) |
| Control | 8.74 | 0 |
| High temperature resistant | 9.24 | 8.39 |
| probiotics | | |

*Control: Inulin coated with probiotics

### Example 10

Heat Resistant Assessment of High Temperature Probiotics According to Present Invention in Milk for Pasteurization.

Pasteurization is the mild heat treatment used in the food industry to eliminate pathogens and extend shelf life of foods and beverages such as milk and fruit juice. In order to use beverage as a probiotic carrier, infusion of probiotics into beverages and subjecting it to pasteurization is necessary for food safety. To serve this purpose, a water barrier layer was spray dispersed on the outer heat resistant layer of high temperature resistant probiotics to prevent infiltration of water when added into beverage.

TABLE 17
Formulation of heat resistant bilayers in
high temperature resistant probiotics

| Seed | Inulin |
|---|---|
| Core layer | Mixture of 100 ml solution containing: |
| | Probiotics Lactobacillus delbrueckii subsp. bulgaricus |
| | Prebiotics: fructooligosaccharides |
| | Binder: whey protein |
| pH- | 200 g Ethanolic solution containing: |
| responsive | Eudragit ® L100 |
| layer | |
| Inner heat | Mixture of 160 g liposome solution containing: |
| resistant layer | Phosphatidylcholine (PC) |
| | Cholesterol |
| | Maltodextrin |
| Outer heat | Mixture of 100 g solution containing: |
| resistant layer | Trehalose |
| | Talc |
| Water barrier | 200 g Ethanolic solution containing: |
| layer | Shellac |

The stability of the encapsulated probiotics during pasteurization in milk as in the industry was evaluated. About 1.37×10⁹ of high temperature resistant probiotics (Formulation in Table 17) was added at 5% w/w into milk and pasteurized at 65° C. for 30 mins in a water bath, followed by rapid cooling in ice bath. Viable counts of probiotics before and after heat treatment was evaluated by first adjusting the pH of the milk to pH 7.4-7.6 and then shaken at 37° C., 120 rpm for 60 min to release the encapsulated probiotics. The mixture was serially diluted and seeded on MRS agar and incubated at 37° C. for 24-48 hours. Probiotics colonies formed on the surface of the agar plate was quantified as cfu/100 ml of sample. Probiotics without encapsulation was used as the control.

Being thermoduric, L. bulgaricus survived pasteurization process. Encapsulation exerted protection to the specific proportion of the heat-treated probiotics. This finding indicate that the coating had shielded a 2.57×10⁵ CFU more probiotics to survive against the heat inactivation process.

TABLE 18
Viable counts of probiotics before and after
milk pasteurization at 65° C. for 30 min

| | Before pasteurization | After pasteurization |
|---|---|---|
| | (CFU in 100 ml) | (CFU in 100 ml) |
| Control | 1.10 × 10⁹ | 1.56 × 10⁶ |
| High temperature resistant | 1.37 × 10⁹ | 2.20 × 10⁶ |
| probiotics | | |

*Control: Inulin coated with probiotics

### Example 11

Heat Resistant Assessment of High Temperature Probiotics According to Present Invention in Orange Juice for Pasteurization.

Natural and fresh orange juice is increasing its demand worldwide. This highly nutritious juice drink is an ideal carrier for probiotics that bring advance health benefits besides vitamins. In order to prolong its shelf-life, pasteurization is used to eliminate the decomposing microorganisms in fruit juice and passivate enzymes that can cause chemical changes. Therefore, infusion of high temperature resistant probiotics into juice prior to pasteurization is necessary to ensure sustainability of live probiotics upon the heat treatment. An additional water barrier was coated on the encapsulated probiotics to keep the encapsulation layers intact and prevent infiltration of water when they were added into beverage.

TABLE 19
Formulation of high temperature resistant probiotics
with additional water barrier layer

| Seed | Inulin |
|---|---|
| Core layer | Mixture of 100 ml solution containing: |
| | Probiotics Lactobacillus delbrueckii subsp. bulgaricus |
| | Prebiotics: fructooligosaccharides |
| | Binder: whey protein |
| pH- | 200 g Ethanolic solution containing: |
| responsive | Eudragit ® L100 |
| layer | |
| Inner heat | Mixture of 160 g liposome solution containing: |
| resistant layer | Phosphatidylcholine (PC) |
| | Cholesterol |
| | Maltodextrin |
| Outer heat | Mixture of 100 g solution containing: |
| resistant layer | Trehalose |
| | Talc |
| Water barrier | 200 g Ethanolic solution containing: |
| layer | Shellac |

The stability of the encapsulated probiotics during pasteurization in juice as in the industry was evaluated. About 100 ml of orange juice containing 2% (w/v; 8.91±0.07 Log CFU) of encapsulated probiotics (Formulation in Table 19) was pasteurized at 65° C. for 30 mins, followed by rapid cooling in ice bath. Viable counts of probiotics before and after heat treatment was evaluated by first adjusting the pH of the orange juice to pH 7.4, followed by shaking at 37° C., 120 rpm for 120 min to release the encapsulated probiotics. The mixture was serially diluted and seeded on MRS agar and incubated at 37° C. for 24-48 hours. Probiotics colonies formed on the surface of the agar plate was quantified as cfu/100 ml of sample. Probiotics without encapsulation was used as the control.

After pasteurization, a 2.37 Log CFU of cells was inactivated from 8.91±0.07 Log CFU, with remaining of 6.54±0.24 Log CFU of high temperature resistant probiotics survived; while control experienced 3.72 Log CFU reduction from 8.29±0.05 Log CFU, contained only 4.57±0.91 Log CFU of probiotics after the heat treatment (Table 20). Infusion of high temperature resistant probiotics into orange juice and pasteurization would ensure delivery of at least 1×10⁶ CFU of probiotics to the gastrointestinal tract and confer health benefits upon consumption.

TABLE 20
Viable counts of probiotics before and after orange
juice pasteurization at 65° C. for 30 min

| | Before pasteurization | After pasteurization |
|---|---|---|
| | (CFU in 100 ml) | (CFU in 100 ml) |
| Control | 8.29 ± 0.05 | 4.57 ± 0.91 |
| High temperature resistant | 8.91 ± 0.07 | 6.54 ± 0.24 |
| probiotics | | |

*Control: Inulin coated with probiotics

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

### REFERENCE

The following references are incorporated herein by reference in their entirety:
Xu et al (2020) Management of corona virus disease-19 (COVID-19): the Zhejiang experience. Zhejiang Da Xue Bao Yi Xue Ban. 49(1):0
Fan Hao, Nan Fu, Hamadel Ndiaye, Meng Wai Woo, Romain Jeantet, Xiao Dong Chen (2020) Thermotolerance, Survival, and Stability of Lactic Acid Bacteria After Spray Drying as Affected by the Increase of Growth Temperature. Food and Bioprocess Technology, 10 (1), pp. 6. ff10.1007/s11947-020-02571-1ff. ffhal-03106934
Gardiner, G. E., E. O'sullivan, J. Kelly, M. A. E. Auty, G. F. Fitzgerald, J. K. Collins, R. P. Ross, And C. Stanton (2020) Comparative survival Rates of Human-Derived Probiotic Lactobacillus paracasei and L. salivarius Strains during Heat Treatment and Spray Drying. Applied and Environmental Microbiology, 66 (6) pp. 2605-2612.
Bernardeau, M., M. J., Lehtinen, S. D. Forssten, P. Nurminen (2017) Importance of the Gastrointestinal Life Cycle of Bacillus for Probiotic Functionality. Journal of Food Science and Technology, 54(8) pp. 2570-2584.

1. A heat and acid resistant probiotics particle having a size from 20 to 250 μm, comprising:
a synbiotic core comprising:
a seed layer comprising at least one polysaccharide; and
a probiotics layer coated on the seed layer;
an acid-resistant shell layer positioned over the synbiotic core, comprising one or more pH-responsive polymers; and
a heat-resistant bilayer shell positioned over the acid-resistant shell layer, comprising:
an inner shell layer comprising a heat-resistant isoprenoid-stabilized phospholipid liposome; and
an outer layer comprising a heat-resistant disaccharide or polysaccharide.

2. The heat and acid resistant probiotics particle of claim 1, wherein the at least one polysaccharide of the seed layer is selected from sucrose, inulin, starch and/or cellulose.

3. The heat and acid resistant probiotics particle of claim 1, wherein the probiotics in the probiotics layer are selected from one or more of Bifidobacterium, Lactobacillus, Lactococcus, Leuconostoc, Streptococcus, Enterococcus, Staphylococcus, Saccharomyces and Kluyveromyces.

4. The heat and acid resistant probiotics particle of claim 1, wherein the probiotics layer further comprises:
one or more of polysaccharides selected from fructo-oligosaccharides, galacto-oligosaccharides, inulin and/or pectin;
one or more of binder protein selected from whey protein, soy protein, chickpea protein, rice protein, pea protein, egg protein, casein, milk protein, zein and/or bovine serum albumin; and/or
one or more of heat-sensitive vitamins selected from vitamin C, vitamin B1 and/or vitamin E.

5. The heat and acid resistant probiotics particle of claim 1, wherein the acid-resistant shell is a pH-responsive polymer selected from one or more of methacrylic acid-methyl methacrylate copolymer, methacrylic acid-ethyl acrylate copolymer, shellac, alginate and/or pectin.

6. The heat and acid resistant probiotics particle of claim 1, wherein the isoprenoid-stabilized phospholipid of the inner shell layer of the heat-resistant bilayer shell is formed by proliposomes comprising:
one or more of phospholipids selected from phosphatidylcholine, phosphatidylethanolamine, phosphatidylinositol, dipalmitoyl phosphatidylcholine, dipalmitoyl phosphatidylethanolamine, dipalmitoyl phosphatidylinositol, dimyristoyl phosphatidylcholine, dimyristoyl phosphatidylethanolamine and/or dimyristoyl phosphatidylinositol;
one or more of isoprenoids selected from β-carotene, cholesterol and/or lycopene; and
one or more of water-soluble carriers selected from maltodextrin, sorbitol, mannitol, maltitol and/or xylitol.

7. The heat and acid resistant probiotics particle of claim 1, wherein the heat-resistant disaccharide or polysaccharide of outer shell layer of the bilayer shell is selected from one or more of sucrose, lactose, maltose, trehalose, cellobiose or chitobiose.

8. The heat and acid resistant probiotics particle of claim 7, wherein the outer shell layer of the bilayer shell further comprises a mineral selected from talc, kaolin, zinc oxide, titanium oxide, silicon oxide, or any combinations thereof.

9. The heat and acid resistant probiotics microsphere of claim 1, wherein the weight percentage of the microsphere comprises 50-79.2% seed, 0.01-0.1% live probiotics, 1.6-3% protein, 2-6.3% polymer, 4-7.9% liposome, equal to or less than 1.6% polysaccharides, equal to or less than 3.2% disaccharides, and equal to or less than 0.3% minerals.

10. The heat and acid resistant probiotics particle of claim 1, further comprising an additional water barrier coating layer coupled to the outer layer including shellac and dimethylaminoethyl methacrylate-copolymer.

11. The heat and acid resistant probiotics particle of claim 1, wherein the particle resists a temperature of up to 90° C. for a period of up to 15 minutes without releasing the probiotics core.

12. A method for preparing a probiotics particle resistant to high temperature and gastrointestinal digestive challenges, the method comprising:
preparing a seed with size between 10-125 μm and density between 0.65 to 0.75 g/cm³ by sieving;
preparing a homogenous live probiotics solution with prebiotics by stir-mixing;
preparing an acid resistant pH-responsive polymer by heating and stir-mixing;
preparing an isoprenoid-stabilized phospholipid liposome by stir-mixing of proliposomes;
preparing a heat resistant outer layer comprising a disaccharide by stir-mixing;
preparing the high temperature resistant probiotics particle with the seed coated with a prebiotic and a probiotic layer, followed by acid resistant layer and heat resistant bilayers by fluidized-bed coating.

13. The method of claim 12, wherein the probiotics particle further comprises a seed core, a probiotic with binder coating on seed surface, an acid resistant protective layer, and a plurality of heat resistant protective layers.

14. The method of claim 12, wherein the probiotics particle has a particle encapsulation efficiency of more than 95%.

15. The method of claim 12, wherein the probiotics particle has a viable count of 10⁸-10¹⁰ CFU/g.

16. The method of claim 12, wherein the probiotics particle has a size of 20-250 μm.

17. The method of claim 12, further comprising infusing the probiotics particle in a food product, a food additive, and liquid beverages.

18. The method of claim 17, wherein the liquid beverages are subjected to thermal treatment including pasteurization.

19. The method of claim 17, wherein the food product further comprises dry food that are subjected to thermal treatment including full baking production process.
